(19) United States
(12) Patent Application Publication
(10) Pub. No.: US 2001/0038079 A1
(43) Pub. Date: Nov. 8, 2001

(54) Method and apparatus for irradiating low energy ion beam on polymers

(76) Inventors: Jang-Ho Ha, Gangbuk-Ku (KR); Byoung-Ho Choi, Seoul (KR); Yong-Sub Cho, Daejeon-Si (KR); Jae-Hyung Lee, Daejeon-Si (KR); Jae-Sang Lee, Daegu-Si (KR); Po-Guk Joo, Seoul (KR)

Correspondence Address: ARENT FOX KINTNER PLOTKIN & KAHN, PLLC, Suite 600, 1050 Connecticut Avenue, N.W., Washington, DC, 20036-5339, US

(21) Appl. No.: 09/790,890

(22) Filed: Feb. 23, 2001

(30) Foreign Application Priority Data
Mar. 6, 2000 (KR) 00-11038

(51) Int. Cl. A61N 5/00; G21G 5/00

(52) U.S. Cl. 250/492.3

(57) ABSTRACT

Disclosed are a method and an apparatus for irradiating low energy ion beam on polymer. The method prepares polymer products having a surface electric conductivity range, in surface electric resistance, from 10⁶ to 10¹¹ Ω/sq, by vacuum-irradiating the ions under relatively low energy of 50-100 keV from ion sources which generates high-current ions of several tens mA or higher, to polymer materials, such as PPO and MPPO, which are electrically insulator; precisely controls temperature so as not to thermally deform molecular configuration of the polymers; produces the products with uniform and stable conductivity in a large area; and treats the surface which improves surface hardness and modifies mechanical properties. Further, the mass-production apparatus for irradiating the ion beams is capable of commercially realizing said method. Accordingly, ions, inert gases (nitrogen, oxygen, argon, xenon, helium, etc.), accelerated at about 50-100 keV are vacuum-irradiated to a depth of 1 μm in polymers (PPO and MPPO), thereby obtaining improved properties of polymers.

### BACKGROUND OF THE INVENTION

1. Field of the Invention

The present invention relates to a method and an apparatus for irradiating low energy ion beam on polymers. More specifically, the present invention relates to a method for producing novel materials having improved physical properties, such as increased electric conductivity and mechanical hardness of polymer surface, by irradiating ions of various elements to polymer materials including PPO (PolyPhenylene Oxide) and MPPO (Modified PolyPhenylene Oxide) which are electric insulator, and an apparatus for mass-producing the modified polymer.

2. Description of the Prior Art

Generally, polymer, which is insulator, having surface electric resistance of 10¹⁵-10¹⁸ Ω/sq or higher, is basic organic materials of various industrial plastics, and comprise covalently bonded monomers which have hydrogen, oxygen, nitrogen, sulfur, fluorine, chlorine and the like linked to carbon chains. Polymer has poor heat resistance because their structure is deformed at about 100-200° C., so that they are difficult to use at high temperature.

Conventional methods for improving electric conductivity of polymeric materials are divided into a method for allowing the polymer itself to have conductivity by chemical mixing and a method for treating only the polymer's surface. In the former method, the polymer is mixed with conductive carbon and metal powder at suitable proportions, molded and then injected. This method increases the conductivity of the polymer's surface proportional to the conductive powder content. However, the products are difficult to prepare and a molded body is easily abraded. The surface electric conductivity may not be uniformly controlled owing to heterogeneous mixing conditions. In addition, the method has the disadvantage of being very brittle, and thus the products manufactured in this way are difficult to recycle, thereby resulting in an increasing environmental contamination.

On the other hand, the treatment method of polymer surface uses low temperature plasma coatings, such as vacuum evaporation and sputturing. This method forms a conductive surface layer of relatively uniform thickness. But this method suffers from inferior adhesion between the conductive layer and the polymer surface, and thus is not practically useful.

Such problems may be alleviated by introducing a novel method for irradiating ions to polymer. This method provides improved electric conductivity and mechanical surface hardness to polymer products by modification of chemical bond configurations of polymer molecules through a collision of irradiated ions with polymer molecules.

Such an irradiation method has the following features; at defined location, depth, thickness of polymer materials, desired surface electric conductivity may be obtained; the surface electric conductivity may be precisely controlled to be made uniform in a wide range (10⁶-10¹¹ Ω/sq); and depth and thickness of the modified layer may be controlled depending on accelerated ion energy.

The degree of bond deformation required to confer electric conductivity and hardness is controlled by an amount of irradiated ions, namely ion numbers. Accordingly, physical properties of the products can be precisely controlled by manipulating accelerating voltage and current of the ion beam apparatus. As necessary, the ion beams are partially and selectively irradiated to only specific regions of polymer materials.

In addition, because only the surface is modified, the products may be recycled after use, whereby this method is environmentally safe.

The reasons why the irradiation method of ion beam is not yet applied to industrial fields are first, that a high-current ion sources of several tens mA or higher is not practically used to an apparatus for generating large quantities of accelerated ions; second, a high brightness ion beam having excellent focusing force for maintaining uniformity of irradiation is difficult to generate; third, deflection and scanning of ion beam for uniform irradiation on large area is difficult to perform; and fourth, preparation costs may not be reduced, even though introduction of simple design concept of the mass-production apparatus.

The limitations of said techniques are surmounted by using high-current ion sources, such as Duoplasmatron or DuoPIGatron, which are designed to produce high-current ion beams, and also have a beam-generating electrode system with excellent focusing force.

Research for improving properties of polymer surfaces by conventional ion implantation has been carried out using high energy ion beams of several hundreds kev-several tens MeV. In particular, for improving hardness of polymer surfaces, a method using high energy ions of 200 keV or higher is disclosed in U.S. Pat. No. 674,840, but is not practically used for large-scale commercial production.

Because a mass production apparatus using high-energy ion beams requires an additional accelerator, the apparatus is complicated and becomes very expensive, and also may not be applied to polymer materials.

Particularly, when high-current ion beams of several tens mA are irradiated to polymers, total kinetic energy of irradiated ions is converted into heat. Also, irradiation of the ion beam is performed in a vacuum so that negligible amount of heat released by conduction through the contact points with black body radiation and material-supporting zone is generated in a heat dissipative process occurring in the irradiated materials.

As such, a heat accumulated to polymer is derived from the following equations;

Q=C·m·ΔT (1)

=V·I·t=q·V·N (2)

where,

Q: total heat accumulated in target

C: specific heat capacity of target

m: material weight

ΔT: temperature change before and after irradiation

V: accelerating voltage

I: current of ion beam

t: irradiating time

e: charge quantity of electron

N: ion beam-irradiated number (ion dose)

In equation (1), if a temperature change (ΔT), a specific heat (C), and a mass (m) are given, total heat (Q) of each polymer material is determined.

Meanwhile, in equation (2), heat generated by ion implantation into polymers is proportional to implanted ion number (N) and energy (eV).

If the number (N) of ions to be implanted is determined, irradiation with minimum possible ion energy is advantageous in mass-producing polymers of given surface conductivity at less than the modification heat thereof.

### SUMMARY OF THE INVENTION

Accordingly, an object of the present invention for alleviating the problems as described above is to provide a method for precisely controlling physical properties of polymer products by manipulating current of ion beams, ion beam energy and irradiation time, for partially modifying the polymer in terms of electric conductivity and mechanical properties, and for precisely controlling the temperature by treating the polymers at low temperature in order not to thermally deform the molecular configuration of the polymers.

Another object of the present invention is to provide an apparatus for uniformly irradiating ions to products of large area by adopting an ion sources not only producing a high-current ion beam of 50 mA or higher but also having excellent ion beam focusing degree, and for irradiating the large quantities of ion beams accelerated immediately after having been produced from the ion sources by use of an electric and magnetic field-generating deflection and scanning system, wherein such simplified ion beam apparatus designed to move a target system in three dimensions for uniform irradiation is advantageous in terms of price competitiveness, and also the mass-production apparatus having a simple arrangement can irradiate the ion beam to a three-dimensional large area or selectively irradiate the ion beams and treat large quantities of products.

In accordance with the present invention, there is provided a method for improving a polymer in mechanical properties and electric conductivity by the irradiation of low energy ion beams onto its surface, wherein ions with an acceleration energy of about 50-100 keV are irradiated from an ion source onto the surface of the polymer, penetrating to a depth of 1 μm under vacuum, thereby changing physical properties of the polymer; ion source uses an inert gases of nitrogen, oxygen, argon, xenon and helium, polymer materials are PPO(polyphenylene oxide) or MPPO(modified polyphenylene oxide), and thus the polymer is suitable for use in antistatic or shield of electromagnetic wave.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a graph showing the relationship between a surface electric resistance and an amount of irradiated nitrogen ion beams at low energy (50 keV). MPPO polymer treated by a method of the present invention.

FIG. 2 is a graph showing the relationship between a mechanical surface hardness and an amount of irradiated nitrogen ion beams of low energy (50 keV) of MPPO polymer treated by a method of the present invention.

FIG. 3 is an elevation of a mass production apparatus for ion beam irradiation for improving electric conductivity of polymeric surface.

FIGS. 4a and 4b are illustrative diagrams of high-current ion sources (Duoplasmatron and DuoPIGatron), capable of producing high-current ion beams of 50 mA or higher, to be applied to the apparatus of the present invention.

FIG. 5 is a diagram of deflection-scanning system of two-dimensional ion beam irradiation which is electrically and magnetically ionized.

FIG. 6 is a diagram of three-dimensional target moving system, capable of carrying out liner motion and rotation motion.

### DETAIL DESCRIPTION OF THE INVENTION

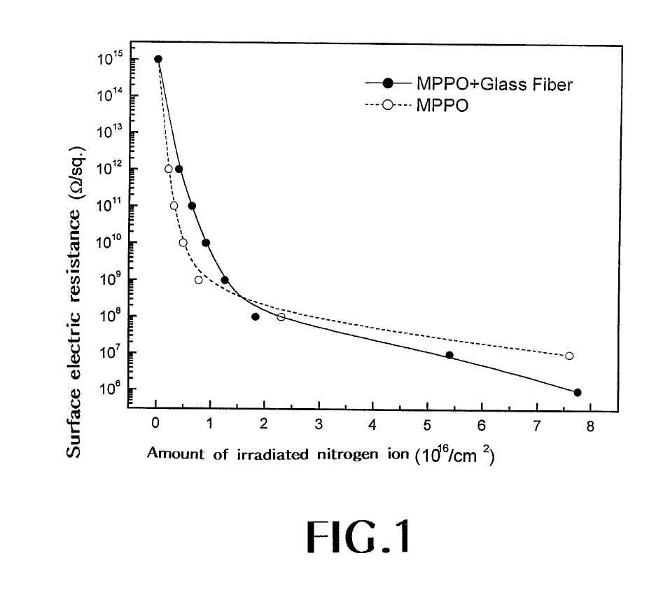

With reference to FIG. 1, there is a graph showing the relationship between surface electric resistance and irradiation amount of nitrogen ions of low energy (50 keV) of MPPO polymer. It is possible to control the irradiation amount of ions in a broad range of 10¹⁴-10¹⁶ ions/cm². In this range, the surface electric resistance of polymers such as MPPO, is drastically decreased to 10⁶-10¹¹ Ω/sq in accordance with an increase of irradiation amount of the ions.

Accordingly, from experimental data shown in FIG. 1, it can be seen that polymer surface conductivity can be precisely controlled by irradiating ion beams while adjusting amounts of ion current generated from ion sources.

When accelerated ions are irradiated onto a insulated polymer, ions incident to the polymer are decelerated until being stopped by repeated collisions with atoms of the polymer, and become diffused within the polymer.

The implanted ions cause molecules of polymer to ionize so that molecular bonds between atoms in the polymer are destroyed and recombined.

Since polymers have lower densities than general metals and inorganic matters, ions have a longer flight range and deeper penetration in polymer than in inorganic matter, long flight range and deep penetration would also be true at high energy. These concepts are independent.

The molecular configuration of polymers is altered in ion beam-penetrated regions, which regions are limited to depths of several μm below surfaces. Thus, a very uniform conductive polymer layer is formed.

For improving surface electric conductivity, helium, nitrogen, argon, xenon and so on, being inert gases, are commonly used. As such, the higher the mass of the irradiated ions is, the more efficient the results obtained are.

Polymers are basically comprised of multiple intermediate molecules, linked by carbon-carbon bonds.

When ions collide with polymers, carbonization, causing surface conductivity, primarily occurs in ion penetration regions, and then temperature is secondarily increased so that modification, decomposition and gas discharge of polymer molecules are generated.

Carbonization means that the implanted ions react with polymer molecules so as to eliminate intermediate molecules and to recombine with carbon atoms themselves.

At that time, the ions implanted into the polymer molecules act as modifiers, which are capable of improving surface electric conductivity.

As a result, surface conductivity is sharply improved near between several millions and several hundred millions of ion irradiation.

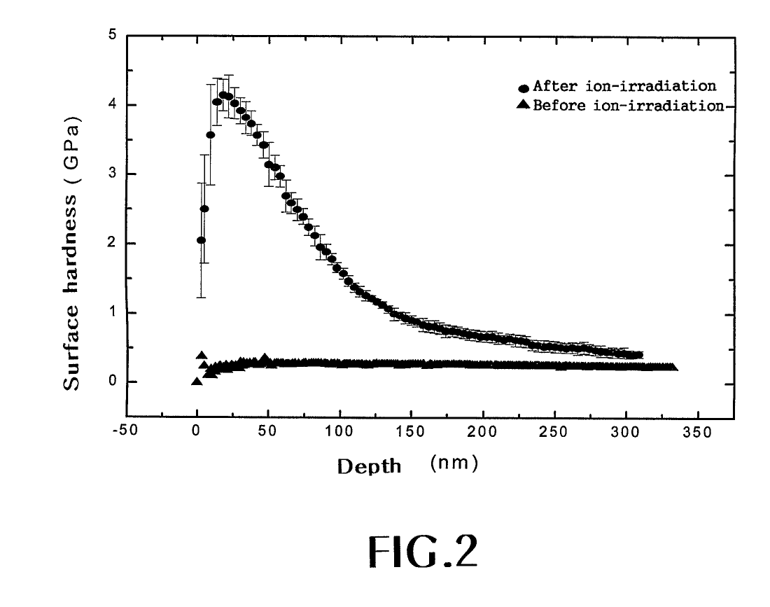

The relationship between mechanical hardness and irradiated amount of nitrogen ions of low energy (50 keV) of MPPO polymer is shown in FIG. 2. From this drawing, it is seen that polymer materials are converted into novel materials having very high mechanical surface hardness.

By irradiating ions, collision of atoms, including carbon and oxygen, results in thermal relaxation and displacement of atomic location. Accordingly, the polymer surface has improved mechanical, chemical and thermal properties.

The reason why the physical properties of the polymer surface are improved is that cross-linking of surface molecules causes the surface to harden by a process of combination-recombination, wherein covalent bonds, such as C—N bonds, between nitrogen ions implanted into the surface and carbons, are newly formed.

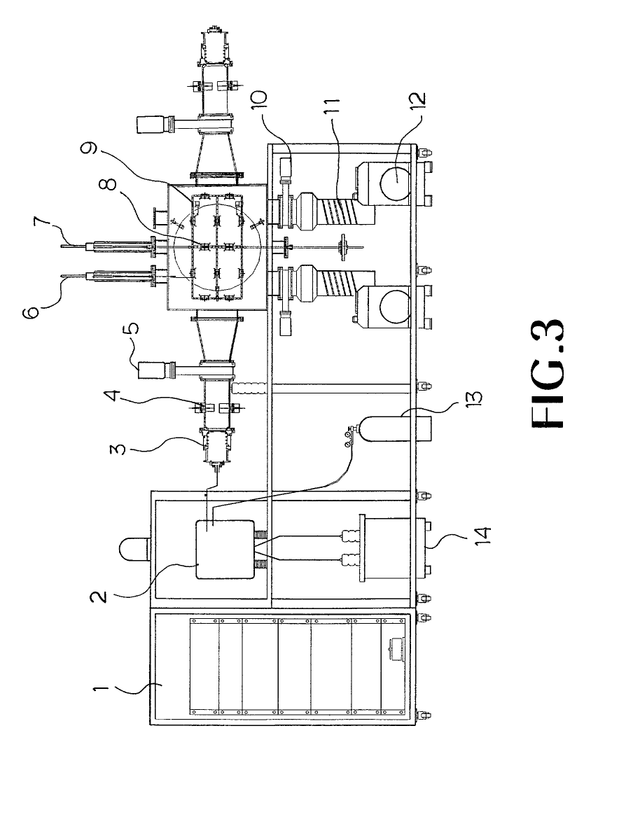

An elevation of a mass production apparatus for irradiating the ion beam used to improve electric conductivity of polymer surfaces is illustrated in FIG. 3. The apparatus of the present invention is different from conventional ion beam apparatuses such as a semiconductor ion injector, in the following aspects; first, the inventive apparatus adopts high-current ion sources having excellent focusing force where ion current is about 50-200 mA, but a conventional apparatus commonly generates ion currents of 10 mA or lower; second, the former is a very simple apparatus accelerating ions using only the power of the ion sources itself, serving as mass production apparatus not requiring the acceleration tube and the mass analyzer, since it uses low energy ions, while the latter is complicated in its arrangement because of having additional acceleration tube and mass analyzers for generating ion beams of high energy; third, the inventive apparatus adopts a simple ion beam deflection-scanning system, capable of simultaneously emitting in two dimensions, by generating electric and magnetic fields in one space at the same time for uniformly irradiating ions to a large area; and fourth, the inventive apparatus adopts a target system, able to carry out linear motion and rotational motion, for irradiating ion beams to polymer in three dimensions.

As illustrated in FIG. 3, the apparatus of the present invention comprises a high vacuum system and apparatus controlling system 1, a high-current ion sources 3, an electric and magnetic deflection scanning system 4, and an ion beam scanning target system 8. Its operation is as follows; desired gas is provided from a gas tank 13 to an ion sources 3 under the control of apparatus-controlling system 1, an ion source power supplying unit is controlled to cause electric discharge inside the ion sources 3, whereby high density of plasma is generated. Thereafter, a high voltage (50-100 kV) is applied to the generated plasma, thereby producing ions.

In order to uniformly irradiate ion beams on target materials of large areas with a minimum of the neutralization caused by combination of generated ions with surrounding electrons, an electric and magnetic field-generating beam deflection-scanning system 4 is located next to the ion sources to simultaneously emit the ion beams in two dimensions, namely horizontally and perpendicularly.

The ion beams thus scanning with a large area can reach targets. In order to be uniformly irradiated with the ion beams, the targets are continuously moved linearly with rotation with the aid of a target transportation and rotation device 7 in combination with rotation devices 31 and 32. The rotation devices 31 and 32 operate to rotate the targets at regular angular intervals so that the targets are uniformly irradiated with the ion beams at predetermined angles.

Uniformity of space distribution of ion beams is measured by an ion beam diagnostic unit 6 utilizing a small Faraday Cup. The ion sources 3 and electric and magnetic deflection scanning system 4 are controlled so that the ion beam is uniformly distributed over a two-dimensional plane.

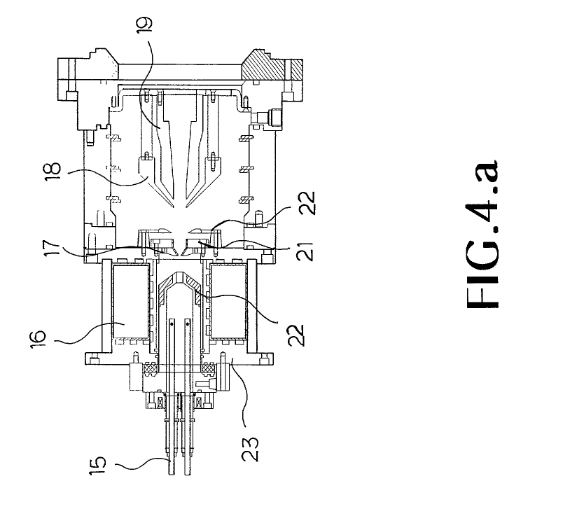

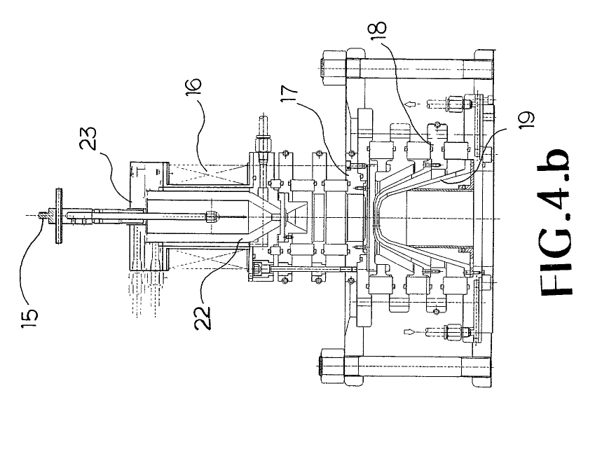

As shown in FIGS. 4a and 4b, high-current ion sources (Duoplasmatron, DuoPIGatron), capable of producing high-current ion beams of 50 mA or higher, are designed to have high-current high brightness beam producing properties. Such Duoplasmatron and DuoPIGatron ion sources are used for the ion beam irradiation apparatus of the present invention.

In the present invention, the beam producing system of Duoplasmatron is improved so that high-current ion beams of several tens mA can be produced and high brightness beams with good focusing degree can be also generated.

High-density plasma emitted from holes of an anode 17 is diluted by use of plasma-expanding cup 21 so that the beam is easily produced. Thereafter, the produced ion beam is accelerated and focused in a connecting electric field configuration where a conical accelerating electrode 18 exists, thereby passing through a decelerating ground electrode 19. The plasma-expanding cup 21 is an important element which determines the brightness of the ion beam, and which plays an important role in defining the contour of the boundary between the plasma and the ion beam and contains a plasma-boundary controlling electrode 20 to which suitable potential is applied. Hence, the contour of the beam-plasma boundary is controlled to produce a high-current high brightness ion beam at all times.

The DuoPIGatron of FIG. 4b is similar to a conventional configuration. An anode 17, an accelerating electrode 18 and a decelerating electrode 19 adopt the conventional beam-producing system with a plurality of holes, but which are formed to have a slit-type configuration suitable for deflection irradiation of the ion beams in the present invention. Accordingly, high-current linear ion beams are scanned so that loss of ions during transportation is reduced and the ion beam is uniformly irradiated to the targets.

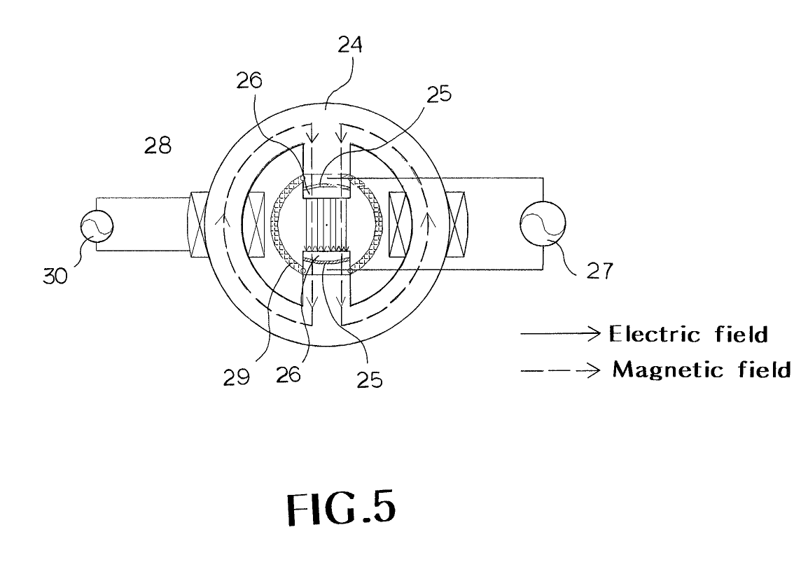

With reference to FIG. 5, there is a deflection and scanning system of ion beam in two dimensions, electrically and magnetically dualized. Such an electric and magnetic hybrid-type two-dimensional ion beam deflection-scanning system allows electric and magnetic fields to be generated so that ion beams are deflected and scanned in horizontal and perpendicular directions.

The high-current ion beam of several tens mA has large diffusion effects because of electric repulsive-force, namely space charge effect, generated among the ions themselves. Therefore, when being spatially diffused, such ions are heterogeneously distributed and thus not controllable. Hence, the targets are not uniformly irradiated with such ion beams.

Therefore, for minimizing the diffusion by such space charge effect of the high-current ion beam, the beam is scanned and then spread over a large area before the ions are diffused, such that the space charge effect may be reduced.

The present invention is characterized in that diffusion and neutralization of ions are reduced by minimizing the travel distance of the ion beam before scanning accomplished by installation of the deflection-scanning system next to the ion sources. Generally, a charge particle beam scanning system operates by an independent diode scanning apparatus which generates electric field or magnetic field in horizontal and perpendicular directions, but suffers from the disadvantage of lengthening a travel path of the ion beam and expanding a size of the second scanning system until the scanning of horizontal and perpendicular directions is completed.

In the present invention, when a saw tooth wave AC voltage is applied from a deflection electrode power unit 27 to an electrode and magnetic pole 26 having an electrode and a magnetic pole jointly, a perpendicular AC electric field is formed so that the ion beam is perpendicularly scanned.

Additionally, the saw tooth wave AC current generated from an electromagnet power unit 30 excites a female coil 28, whereby a magnetic field is generated between poles 26 through a magnetic circuit 24 comprising ferromagnetic substances. Hence, the ions are scanned in a horizontal direction.

To electrically insulate the both poles 26, an insulating ferrite 25 which has not only large magnetic permeability but also high electric resistance is used.

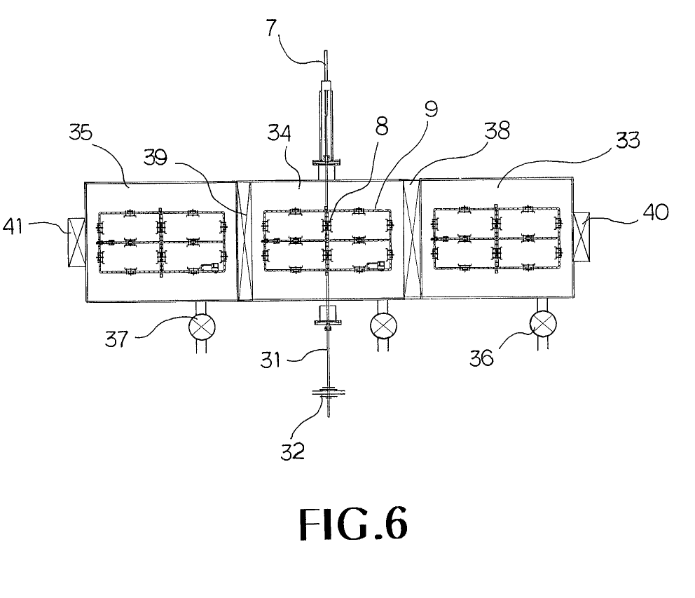

FIG. 6 shows a three-dimensional target system, capable of carrying out linear motion and rotational motion. Also, this drawing is a block diagram of a target system for irradiating and treating large quantities of various planar polymers.

A vacuum chamber comprises a front chamber 33, a target irradiation chamber 34 and a rear chamber 35, in which the irradiation chamber is designed to carry out rotational and linear motions of the targets by the rotation devices 31 and 32 and the target transportation and rotation device 7 so as to uniformly irradiate various shapes of targets. The targets are placed in the front chamber, after which air is evacuated from the front chamber by use of a front chamber vacuum valve 36. Then, the targets are transferred to the target irradiation chamber 34 after opening a front chamber gate valve 38, and then irradiated with the ion beam while being moved with the aid of the linear and rotational motion devices until the irradiation energy of the ion beam reaches a desired level.

With opening a rear chamber gate valve 39, the products are transferred to the rear chamber, and then let out into atmosphere after opening a target outlet 41, whereby the products can be treated in large scale by efficient continuous processes while connecting the atmosphere and a high vacuum system.

Accordingly, the apparatus of the present invention can be used to produce high-current ion beams of 50 mA or higher for irradiation-treating large quantities of polymer materials, to uniformly irradiate the ion beams having excellent focusing degree to products of large area, to simultaneously produce and accelerate the ions from only ion source, and to irradiate the accelerated ion beam onto products of large area by the electric and magnetic field-generating deflection-scanning system. Such simplified ion beam apparatus is advantageous in terms of price competitiveness. In addition, for securing uniformity of ion beam irradiation, the targeting system may be moved in three dimensions by use of the inventive apparatus. Use can be made of the mass-production apparatus for three-dimensionally irradiating the ion beam onto polymer materials of large area by manipulating current of ion beams, ion beam energy and irradiation time, for partially modifying the polymer in electric conductivity and mechanical properties, and for precisely controlling the temperature by treating the polymers at low temperature in order not to thermally deform the molecular configuration of the polymers. Thusly obtained polymer is suitable for use in antistatic or electromagnetic wave-shield fields.

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

What is claimed is:

1. A method for improving a polymer in mechanical properties and electric conductivity by the irradiation of low energy ion beams onto its surface, wherein ions with an acceleration energy of about 50-100 keV are irradiated from an ion source onto the surface of the polymer to a depth of 1 μm under vacuum, thereby changing physical properties of the polymer, said ion source is an inert gas selected from the group consisting of nitrogen, oxygen, argon, xenon and helium, polymers being polyphenylene oxide or modified polyphenylene oxide.

2. The method as set forth in claim 1, wherein the polymer has a surface electric conductivity range, in surface electric resistance, from 10⁶ to 10¹¹ Ω/sq.

3. The method as set forth in claim 1, wherein the surface electric conductivity is precisely controlled by modulating an ion beam current generated from the ion source upon the irradiation.

4. The method as set forth in claim 1, wherein the polymer has uniform and stable surface conductivity on its large surface area and is improved in strength, hardness and mechanical properties.

5. The method as set forth in claim 1, wherein the polymer is suitable for use in antistatic or electromagnetic wave-shield fields.

6. An apparatus for improving a polymer in mechanical properties and electric conductivity by the irradiation of low energy ion beams onto its surface, the apparatus being characterized in that desired gas is provided from a gas tank to an ion source under the control of an apparatus-controlling system, then, an ion source power supplying unit being controlled to cause electric discharge inside the high-current ion source and thus high density of plasma being generated, a high voltage (50-100 kV) being applied to the generated plasma, and an electric and magnetic field-generating beam deflection-scanning system located next to a high-current ion source allows the ion beams to simultaneously scan in horizontal and perpendicular directions, the targets being continuously moved linearly with rotation with the aid of a target transportation and rotation device in combination with rotation devices to rotate the targets at regular angular intervals so that the targets are uniformly irradiated with the ion beams at predetermined angles, the ion sources and the deflection-scanning system are controlled so that the ion beam is uniformly distributed over a two-dimensional plane, using an ion beam diagnostic unit.

7. The apparatus as set forth in claim 6, the apparatus being characterized in that a high-density plasma from holes of an anode is diluted by use of a plasma-expanding cup so that the beam is easily produced, after which the produced ion beam is accelerated and focused in a connecting electric field configuration where a conical accelerating electrode exists, thereby passing through a decelerating ground electrode, said plasma-expanding cup being able to define the contour of the boundary between the plasma and the ion beam, and containing a plasma-boundary controlling electrode to which suitable potential is applied, whereby the beam-plasma boundary is controlled to produce a high-current high brightness ion beam.

8. The apparatus as set forth in claim 6, the apparatus being characterized in that the anode, an accelerating electrode and a decelerating electrode which produce ion beams, have slit-type configuration suitable for deflection and irradiation of the ion beams.

9. The apparatus as set forth in claim 6, the apparatus being characterized in that the electric and magnetic field-generating deflection-scanning system is located at a rear end of the high-current ion source, whereby neutralization of high-current ion beams is minimized and ion beams are uniformly irradiated onto two-dimensional large areas.

10. The apparatus as set forth in claim 6, the apparatus being characterized in that targets are placed in a front chamber, the air being evacuated from the front chamber by use of a front chamber vacuum valve, then, the targets being transferred to a target irradiation chamber after opening a front chamber gate valve and then irradiated with the ion beam while being moved with the aid of the linear and rotational motion devices until the irradiation energy of the ion beam reaches a desired level, the targets being transferred to a rear chamber with opening of a rear chamber gate valve, and then let out into atmosphere after opening a target outlet 41, whereby the ion beams are uniformly irradiated and the targets are linearly and rotationally moved for three-dimensional irradiation.

11. The apparatus as set forth in claim 6, the apparatus being characterized in that a plural number of ion sources, capable of simultaneously irradiating ion beam to both sides of the polymer, are mounted.
